(19) United States
(12) Patent Application Publication
(10) Pub. No.: US 2026/0309680 A1
(43) Pub. Date: Oct. 8, 2026

(54) ULTRASOUND IMAGING DEBRIS FILTER SYSTEM AND METHODS

(71) Applicant: Ulthera, Inc., Raleigh, NC (US)

(72) Inventors: Stephen J. Hsu, Raleigh, NC (US); Andrey Varvarkin, Kirkkonummi (FI); Vladimir Milevski, St. Petersburg (RU); Joseph W. R. Howse, Terence Bay (CA)

(21) Appl. No.: 19/680,802

(22) Filed: May 18, 2026

Related U.S. Application Data
(63) Continuation of application No. PCT/US2024/047440, filed on Sep. 19, 2024.
(60) Provisional application No. 63/571,760, filed on Mar. 29, 2024.

(51) Int. Cl. G01N 29/44; G01N 29/06; G01N 29/22

(52) U.S. Cl. CPC G01N 29/4463; G01N 29/0609; G01N 29/0654; G01N 29/226

(57) ABSTRACT

Systems and methods for filtering debris from an ultrasonic image of an ultrasonic imaging system. Methods may include receiving an ultrasonic image of a plurality of ultrasonic images, determining a zone of the ultrasonic image, applying a mask to the zone of the ultrasonic image, wherein the mask is applied to the zone for a first number of iterations, determining a level of noise in the ultrasonic image, determining, based on the level of noise, that additional noise suppression is needed, applying the mask to the zone of the ultrasonic image, wherein the mask is applied to the zone for a second number of iterations, and displaying the ultrasonic image on a display.

### PRIORITY AND RELATED APPLICATIONS

The present application is a continuation of PCT App. No. PCT/US2024/047440 filed on Sep. 19, 2024, which claims the benefit of U.S. Provisional Application No. 63/571,760 filed on Mar. 29, 2024, entitled ULTRASOUND IMAGING DEBRIS FILTER SYSTEM AND METHODS. Any and all applications for which a foreign or domestic priority claim is identified in the Application Data Sheet as filed with the present application are hereby incorporated by reference under 37 CFR § 1.57.

### BACKGROUND

### Field

The present disclosure relates to the field of imaging tissue, using, for example, ultrasound. Several embodiments of the invention relate to systems and methods of reducing noise that may appear on ultrasound images of an ultrasonic imaging system, such as noise caused by debris.

### Description of the Related Art

Conventional techniques for debris removal from ultrasonic images may include physical removal of debris from the ultrasonic imaging device.

### SUMMARY

There is a need for improved resolution for ultrasound imaging to quickly, efficiently, and accurately image tissue for aesthetic and/or cosmetic treatments of skin and/or tissue underlying the skin. In several embodiments, an ultrasound system is configured for imaging to visualize tissue (e.g., epidermal, dermal and/or subdermal, fascia, muscle, fat, and other tissues). In various embodiments, an ultrasound system is configured for imaging to visualize tissue to confirm appropriate depth of an associated cosmetic or medical treatment such as to avoid certain tissues (e.g., nerve, bone, etc.).

In several embodiments, the present disclosure relates to a system of ultrasound imaging. In some embodiments, the system is configured for ultrasound therapy. In some embodiments, the system includes both ultrasound imaging and therapy. Ultrasound imaging systems may include various components, such as an imaging element and a display, to visualize a tissue region and/or anatomy of the body. In addition, a coupling medium, such as a gel or a fluid (e.g., water, saline) can be used to allow acoustic waves from the imaging element to pass into the tissue region without disruption. Debris, air bubbles, and/or other materials between the imaging element and the tissue region can interfere with the imaging and ultimately affect the image captured by the ultrasound imaging system. This may result in speckles or artifacts that can appear on the ultrasonic image. Alternatively, or in addition, noisy images can result from deterioration and aging of the imaging element over time.

Several embodiments of the present disclosure relate to systems and methods of digital noise suppression for ultrasonic imaging systems. For example, a digital filter algorithm may be used to suppress debris noise that can appear on ultrasound images. In one embodiment, an ultrasonic image may be partitioned via algorithm into discrete zones, over which noise suppression filters may be passed. In one embodiment, the systems and methods described herein can determine whether additional iterations, or passes, of a debris filter algorithm can be used to further suppress noise.

Systems and methods for ultrasound imaging of tissue are adapted for and/or configured to use one or more digital filter algorithms to help to suppress debris noise in ultrasound images in several embodiments. An ultrasound transducer for imaging may have an offset gap between the imaging transducer and a portion of a housing (such as at a window, such as a PEEK window) of an ultrasound probe, whereby the portion of the housing is placed in contact through acoustic coupling to a tissue such as a skin surface for imaging the one or more focal zones under the skin surface. In some embodiments, an ultrasound transducer for imaging has an offset gap between the imaging transducer and a portion of a housing that uses two or more (e.g., 2, 3, 4, 5, 6, or more) focal zones that can produce debris artifacts from acoustic ultrasound energy that bounces between the imaging transducer and (i) the acoustic window and/or (ii) the region being imaged. These debris artifacts may obscure the clarity of the imaging. In various embodiments described herein, systems and methods reduce and/or eliminate such artifacts and to improve ultrasound image quality.

In various embodiments, ultrasound imaging is used to visualize a tissue region and/or anatomy. In one embodiment, ultrasound imaging is used to confirm sufficient acoustic coupling to a tissue region for improving imaging correlation between movement of the ultrasound imaging transducer in a first and second direction when forming images. In various embodiments, ultrasound imaging is used in conjunction with a cosmetic treatment or a medical treatment in order to visualize, plan and/or monitor the cosmetic or medical treatment. Ultrasound imaging may be used in conjunction with an application of energy to a tissue. Ultrasound imaging can be used in conjunction with an application of ultrasound therapy to a tissue. In several embodiments, ultrasound imaging is used in conjunction with an application of a dermal filler to a tissue. Ultrasound imaging and/or therapy may be used in conjunction with an application of a drug or a compound to a tissue. Ultrasound imaging can be used in conjunction with an application of a botulinum toxin to a tissue. In various embodiments, an ultrasound system may be configured for focusing ultrasound to produce localized, mechanical motion within tissues and cells for the purpose of producing either localized heating for tissue coagulation or for mechanical cellular membrane disruption intended for non-invasive aesthetic use. In various embodiments, an ultrasound system can be configured for lifting a brow (e.g., an eyebrow) and/or for lifting lift lax tissue, such as submental (beneath the chin) and neck tissue. In various embodiments, an ultrasound system is configured for improving lines and wrinkles of the décolleté. An ultrasound system can be configured for reducing fat. In various embodiments, an ultrasound system is configured for reducing the appearance of cellulite. In several embodiments disclosed herein, non-invasive ultrasound systems are adapted to be used in achieving one or more of the following beneficial aesthetic and/or cosmetic improvement effects: a face lift, a brow lift, a chin lift, an eye treatment (e.g., malar bags, treat infraorbital laxity), a wrinkle reduction, fat reduction (e.g., treatment of adipose and/or cellulite), cellulite (which may be called gynoid lipodystrophy) treatment (e.g., dimple or non-dimple type female gynoid lipodystrophy), décolletage improvement (e.g., upper chest), a buttock lift (e.g., buttock tightening), skin tightening (for example, treating laxity to cause tightening on the face or body, such as the face, neck, chest, arms, thighs, abdomen, buttocks, etc.), a scar reduction, a burn treatment, a tattoo removal, a vein removal, a vein reduction, a treatment on a sweat gland, a treatment of hyperhidrosis, a sun spot removal, an acne treatment, a pimple reduction.

Several embodiments are particularly advantageous because they include one, several or all of the following benefits: (i) higher imaging resolution, (ii) removal of obscuring artifacts and debris from imaging, (iii) clearer imaging using a moving imaging transducer, (iv) more efficient imaging, and/or (v) improved imaging to assist in associated treatment or therapy.

In several embodiments, a method for filtering debris from an ultrasonic image includes receiving a frame of an ultrasonic image from an ultrasound imaging system; determining that the frame meets a contact threshold; applying a first mask to a zone of the frame, wherein the zone corresponds to a portion of the frame at a first depth range; determining, within the zone, a difference between the frame and frame with the first mask applied; applying an edge detection filter to the zone of the frame; determining an average intensity of the zone, wherein the average intensity corresponds to an estimated level of noise within the zone; applying, based on the estimated level of noise in the zone, a second mask to the zone to produce a noise suppressed zone of the frame; and displaying the frame on a display of the ultrasound imaging system.

In one embodiment, the contact threshold corresponds to an intensity distribution of the frame. The first mask and the second mask can be noise suppression masks. The method can include normalizing the estimated level of noise in the zone. In one embodiment, the applying of the edge detection filter causes a gradient to be output. The method can include determining that additional noise suppression is needed based on the estimated level of noise and applying additional iterations of the second mask to the zone.

In one embodiment, application of the first or second mask includes determining, for each pixel of the zone of the frame, whether an input pixel value is equal to a result of a horizontal maximum filter; determining an output pixel value based on a weighted median filter to the pixel in response to the determination that the input pixel value is equal to the result of the horizonal maximum filter; and determining an output pixel value based on an average of the horizontal maximum filter and a horizontal minimum filter in response to the determination that the input pixel value is not equal to the result of the horizonal maximum filter.

In several embodiments, an ultrasound imaging system configured for digitally filtering debris from an ultrasonic image includes an ultrasonic probe comprising an ultrasound imaging transducer adapted for imaging a tissue region; a display coupled to the ultrasonic probe; and a processor coupled to the ultrasonic probe and configured to: receive a frame of an ultrasonic image from an ultrasonic imaging system; determine that the frame meets a contact threshold; apply a first mask to the frame, determine an absolute difference between the frame and frame with the first mask applied; apply an edge detection filter to the frame; determine an average intensity of the frame, wherein the average intensity corresponds to an estimated level of noise within the frame; apply, based on the estimated level of noise, a second mask to the frame to produce a noise suppressed frame; and display the frame on a display of the ultrasonic imaging system.

In one embodiment, the contact threshold corresponds to an intensity distribution of the frame. The first mask and the second mask can be noise suppression masks. The processor can be further to normalize the estimated level of noise in the frame. In one embodiment, the applying of the edge detection filter causes a gradient to be output. The processor can be further configured to determine that additional noise suppression is needed based on the estimated level of noise; and apply additional iterations of the second mask to the frame. The application of the first or second mask can further cause the processor to: determine, for each pixel of the frame, whether an input pixel value is equal to a result of a horizonal maximum filter; determine an output pixel value based on a weighted median filter to the pixel in response to the determination that the input pixel value is equal to the result of the horizontal maximum filter; and determine an output pixel value based on an average of the horizontal maximum filter and a horizontal minimum filter in response to the determination that the input pixel value is not equal to the result of the horizonal maximum filter.

In several embodiments, an ultrasound imaging system configured for digitally filtering debris from an ultrasonic image includes an ultrasonic probe comprising an ultrasound imaging transducer adapted for imaging a tissue region; a display coupled to the ultrasonic probe; and a processor coupled to the ultrasonic probe and configured to: apply a first filter to an ultrasonic image, determine an absolute difference between the ultrasonic image and ultrasonic image with the first filter applied; applying an edge detection filter to the ultrasonic image; determining an intensity of the ultrasonic image, wherein the intensity corresponds to an estimated level of noise of the ultrasonic image; applying, based on the estimated level of noise, a second filter to a frame to produce a noise suppressed ultrasonic image; and displaying the noise suppressed ultrasonic image on a display of the ultrasonic imaging system.

The first filter and the second filter can be noise suppression filters. In one embodiment, the processor is further to normalize the estimated level of noise. The application of the edge detection filter can cause a gradient to be output. The processor can further be configured to determine that additional noise suppression is needed based on the estimated level of noise; and apply additional iterations of the second filter to the frame. In one embodiment, the application of the first or second filter further causes the processor to: determine, for each pixel of the frame, whether an input pixel value is equal to a result of a horizonal maximum filter; determine an output pixel value based on a weighted median filter to the pixel in response to the determination that the input pixel value is equal to the result of the horizontal maximum filter; and determine an output pixel value based on an average of the horizontal maximum filter and a horizontal minimum filter in response to the determination that the source pixel value is not equal to the result of the horizonal maximum filter. In some embodiments, the system comprises various features that are present as single features (as opposed to multiple features). Multiple features or components are provided in alternate embodiments. In various embodiments, the system comprises, consists essentially of, or consists of one, two, three, or more embodiments of any features or components disclosed herein. In some embodiments, a feature or component is not included and can be negatively disclaimed from a specific claim, such that the system is without such feature or component. In some embodiments, a method is performed without a step. In some embodiments, a system does not comprise a certain component. Further, areas of applicability will become apparent from the description provided herein. It should be understood that the description and specific examples are intended for purposes of illustration only and are not intended to limit the scope of the embodiments disclosed herein.

### BRIEF DESCRIPTION OF THE DRAWINGS

Various features will now be described with reference to the following drawings. Throughout the drawings, reference numbers may be re-used to indicate correspondence between referenced elements. The drawings are provided to illustrate examples described herein and are not intended to limit the scope of the disclosure. Embodiments will become more fully understood from the detailed description and the accompanying drawings. Features from one drawing are applicable to other drawings in several embodiments.

FIG. 1 is a schematic illustration of an ultrasound system according to various embodiments.

FIG. 2 is a schematic illustration of an ultrasound system according to various embodiments.

FIG. 3 is a schematic illustration of an ultrasound system according to various embodiments.

FIG. 4 illustrates an example block diagram of an image processing system within the ultrasonic imaging system, according to various embodiments of the present disclosure.

FIG. 5 illustrates an example block diagram of components of the image processing system to process an ultrasonic image captured by the ultrasound imaging system according to various embodiments of the present disclosure.

FIGS. 6A-6C illustrate a sample ultrasonic image that may be processed by the image thresholding system, according to various embodiments of the present disclosure.

FIGS. 7A-7E illustrate sample ultrasonic images that may be processed by the noise suppression system and the noise estimation system, according to various embodiments of the present disclosure.

FIG. 8 is an example flow diagram of debris detection and filtering logic utilized by the image processing system according to various embodiments of the present disclosure.

FIG. 9 is a table illustrating example filter aperture sizes according to various embodiments of the present disclosure.

FIG. 10 is a table illustrating example filters and corresponding depth ranges and iterations according to various embodiments of the present disclosure.

FIG. 11 is an example scheme of filter sizes and iterations used by the image processing system at corresponding zones of an ultrasonic image according to various embodiments of the present disclosure.

FIG. 12 is an example scheme of filter sizes and iterations used by the image processing system at corresponding zones of an ultrasonic image containing weak debris noise according to various embodiments of the present disclosure.

FIGS. 13A-B are example flow diagrams of a routine to digitally filter noise debris from an ultrasonic image of an ultrasound imaging system according to various embodiments of the present disclosure.

### DETAILED DESCRIPTION

The following description sets forth examples of embodiments of systems and methods associated with ultrasound imaging and is not intended to limit the present invention or its teachings, applications, or uses thereof. It should be understood that throughout the drawings, corresponding reference numerals indicate like or corresponding parts and features. The description of specific examples indicated in various embodiments are intended for purposes of illustration only and are not intended to limit the scope of the invention disclosed herein. Moreover, recitation of multiple embodiments having stated features is not intended to exclude other embodiments having additional features or other embodiments incorporating different combinations of the stated features. Further, features in one embodiment (such as in one figure) may be combined with descriptions (and figures) of other embodiments.

Noise, speckle noise, or artifact noise (collectively, noise) can be a property of ultrasound imaging that generally tends to reduce the image resolution or contrast of an ultrasound image. Ultimately, this decrease in resolution or contrast may reduce the diagnostic value of the ultrasound image(s). In some cases, noise may result from the buildup of debris and other obstructions one or around the ultrasound imaging element (“debris noise”). In some cases, debris noise may result from the buildup of debris and other obstructions between the ultrasound imaging element and a target region for imaging or treatment. For example, air bubbles, dirt, and other materials within the coupling medium may obstruct the ultrasound imaging element from capturing a clean ultrasound image. Within an image, debris noise may be characterized by vertically and obliquely directed curves and lines of high intensity. In several embodiments, these lines usually have a projection on the X-axis that is significantly smaller than the projection on the Y-axis. As such, the horizontal cross-section of these high-intensity lines typically consists of no more than a few pixels.

In several embodiments, median filtering can be used to address the issue of noise in ultrasonic and other types of imaging. This is a filter in which each output pixel is computed as the median value of the input pixels in the aperture. Median filters may suppress impulse noise that manifests in an image as bright and dark pixels that may appear randomly throughout the spatial distribution. The result of this filtering includes smoothing and shifting of object boundaries, which may result in the reduction of small objects that appear in the image. In order to suppress high-intensity debris noise lines using median filtering, it would be advantageous to utilize an aperture filter size adapted to the width of the high-intensity noise debris lines (e.g., the projection on the X-axis). The debris noise filtering system described herein may be configured to suppress debris noise resulting from the buildup of debris and other obstructions around the ultrasound imaging element. An indicator of suspected debris presence in an image row includes an output value of the horizontal Max filter equal to the pixel value of the input image. In this case, the result of the filter will be the average value between the minimum filter of the same aperture and the median filter. Using the minimum filter may accelerate the convergence of the filtering procedure. If no debris is detected, the result of noise suppression includes an average value between the pixel of the original image and the result of applying the weighted median filter. This allows for light image filtering and may reduce the difference between pixels processed in different branches.

In addition, the systems described herein may be configured to execute an iterative filtering procedure. As noted herein, in one embodiment, the upper part of an ultrasonic image very rarely contains debris. Therefore, the top part (5-10% in depth from the top) is usually not subjected to filtering. The middle part of the ultrasonic image typically contains weak debris with a small cross-section along the X-axis. Therefore, the system may filter the middle part is filtered with a 3-pixel aperture size filter. As the depth increases, the debris noise is typically stronger and thicker. Accordingly, the bottom part of the ultrasonic image may be filtered by several passes of 3×3 filter (“F3”) and several passes of a 5×5 filter (“F5”) according to various embodiments.

Further, the systems as described herein relate to noise level assessment and filter parameter setting. To assess noise and set filter parameters, filters used to suppress debris noise are used by the system. In some embodiments, the system will perform noise level assessment once every N frames. The frame may first be input into an image threshold system. This may ensure that the noise assessment is based on a real ultrasound image. This allows the system to avoid situations when a sensor of the ultrasound imaging system is pointed into the air or has poor contact or poor acoustic coupling with a patient's body. The image thresholding system analyzes the distribution of ultrasound image intensity across width and depth, symmetry across width, and some other features. If the image is determined to have sufficient contact, further image processing may be conducted.

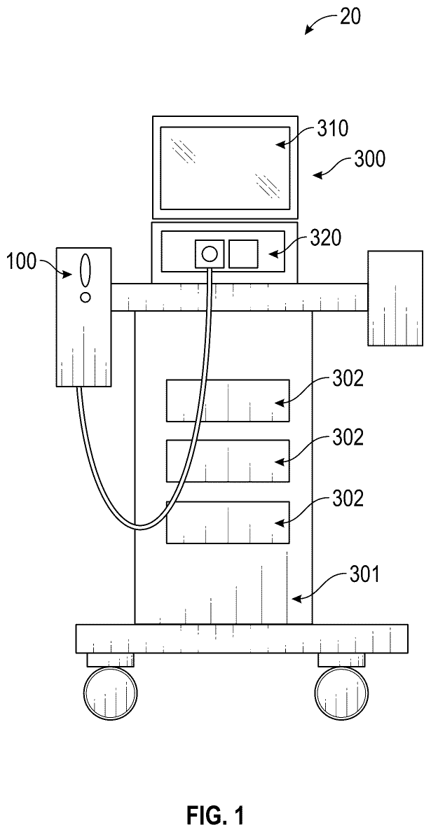

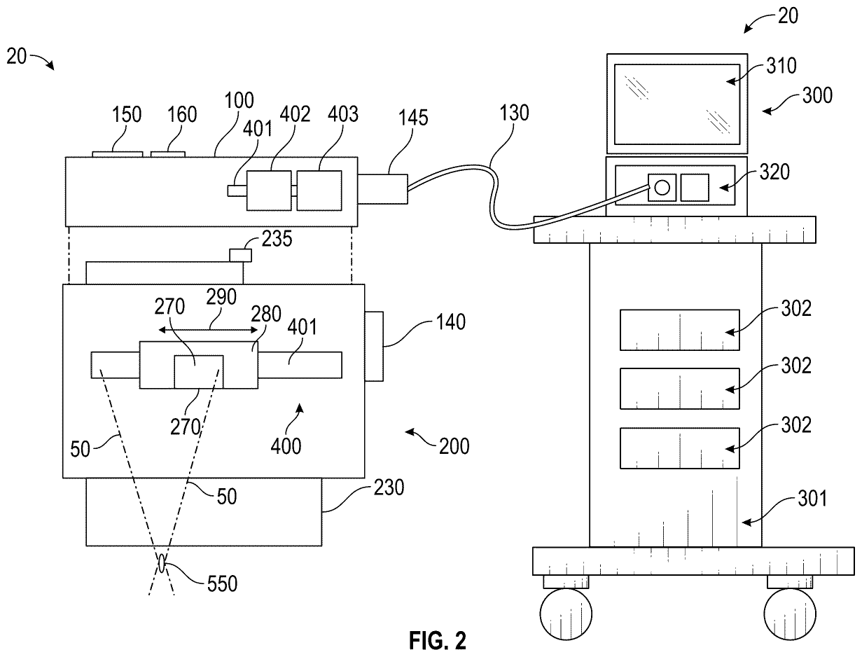

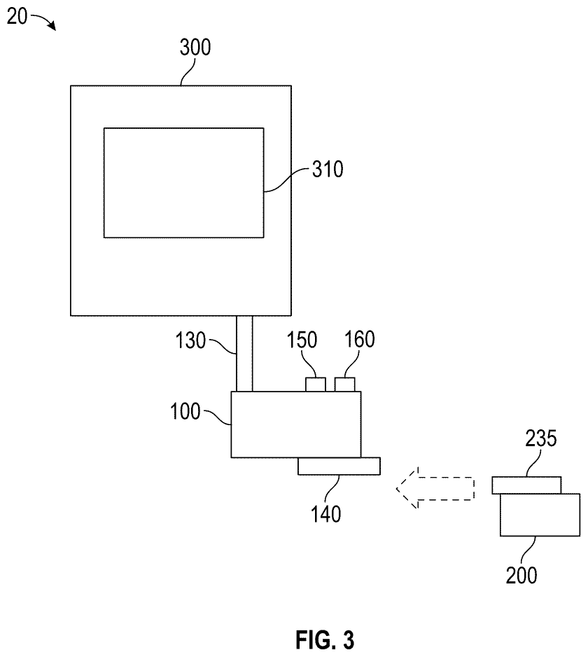

FIGS. 1-3 illustrate an example ultrasound imaging system 20 configured to suppress debris noise from ultrasonic images, according to various embodiments of the present disclosure.

In some embodiments, ultrasound imaging system 20 includes a hand wand (e.g., handpiece) 100, module (e.g., transducer module, cartridge, probe) 200, and a console 300. In several embodiments, the console 300 comprises a controller 305. In some embodiments, a console 300 comprises a communication system (e.g., Wi-Fi, Bluetooth, modem, etc. to communicate with another party, a manufacturer, a supplier, a service provider, the Internet, and/or a cloud. In various embodiments, the console 300 comprises a metal housing (e.g., Magnesium, Aluminum, Titanium, and alloys). In some embodiments, a cart 301 provides mobility and/or position of the system 20, and can include wheels, surfaces to write on or place components, and/or compartments 302 (e.g., drawers, containers, shelves, etc.) to, for example, store or organize components. In some embodiments, the cart has a power supply, such as a power connection to a battery and/or one or more cords to connect power, communications (e.g., internet, Ethernet, etc.) to the system 20. In some embodiments, the system 20 comprises a cart 301. In some embodiments, the system 20 does not comprise a cart 301.

The hand wand 100 can be coupled to the controller 305 by an interface 130, which may be a wired (e.g., cable) or wireless (e.g., Bluetooth, Wifi, etc.) interface. The interface 130 can be coupled to the hand wand 100 by a mechanical and/or electrical connector 145. The distal end of the interface 130 can be connected to a controller connector on a circuit 345 (not shown). In one embodiment, the interface 130 can transmit controllable power from the controller 305 to the hand wand 100. In one embodiment, the system 20 has one or more imaging channels (e.g., 1, 2, 4, 6, 8, 10 channels) for ultra-clear HD (high definition) visualization of subcutaneous structures to improve imaging. In an embodiment, the system 20 has one or more therapy channels (e.g., 1, 2, 4, 6, 8, 10 channels) and a precision linear-drive motor that doubles treatment accuracy while increasing speed (e.g., by 25%, 40%, 50%, 60%, 75%, 100% or more).

In various embodiments, the controller 305 can be adapted to and/or configured for operation with the hand wand 100 and the module 200, as well as the overall ultrasound system 20 functionality. The controller 305 can include connectivity to one or more interactive graphical display 310, which can include a touchscreen monitor and Graphic User Interface (GUI) that allows the user to interact with the ultrasound system 20. In one embodiment, a second smaller, more mobile display allows the user to more easily position and view the treatment screen. In one embodiment, a second display allows the system user to view a treatment screen (e.g., on a wall, mobile device, large screen, remote screen). In one embodiment the graphical display 310 includes a touchscreen interface 315 (not shown). In various embodiments, the display 310 sets and displays the operating conditions, including equipment activation status, treatment parameters, system messages and prompts, and ultrasound images.

In various embodiments, the controller 305 can be adapted to and/or configured to include, for example, a microprocessor with software and input/output devices, systems and devices for controlling electronic and/or mechanical scanning and/or multiplexing of transducers and/or multiplexing of transducer modules, a system for power delivery, systems for monitoring, systems for sensing the spatial position of the probe and/or transducers and/or multiplexing of transducer modules, and/or systems for handling user input and recording treatment results, among others. In some embodiments, the controller 305 may contain a processor, not shown, configured to execute instructions stored in memory to suppress noise of an ultrasonic image. For example, the processor may be configured to execute processes of an image processing system 401 to perform noise suppression on an ultrasonic image captured by ultrasound imaging system 20.

In various embodiments, the controller 305 can include a system processor and various analog and/or digital control logic, such as one or more of microcontrollers, microprocessors, field-programmable gate arrays, computer boards, and associated components, including firmware and control software, which may be capable of interfacing with user controls and interfacing circuits as well as input/output circuits and systems for communications, displays, interfacing, storage, documentation, and other useful functions. System software running on the system process may be adapted to and/or configured to control all initialization, timing, level setting, monitoring, safety monitoring, and all other ultrasound system functions for accomplishing user-defined treatment objectives. Further, the controller 305 can include various input/output modules, such as switches, buttons, etc., that may also be suitably adapted to and/or configured to control operation of the ultrasound system 20.

In one embodiment, the hand wand 100 includes one or more finger activated controllers or switches, such as 150 and 160. In various embodiments, one or more thermal treatment controllers 160 (e.g., switch, button) activates and/or stops treatment. In various embodiments, one or more imaging controllers 150 (e.g., switch, button) activates and/or stops imaging. In one embodiment, the hand wand 100 can include a removable module 200. In other embodiments, the module 200 may be non-removable. In various embodiments, the module 200 can be mechanically coupled to the hand wand 100 using a latch or coupler 140. In various embodiments, an interface guide 235 or multiple interface guides 235 can be used for assisting the coupling of the module 200 to the hand wand 100. The module 200 can include one or more ultrasound imaging transducers 270. In some embodiments, an ultrasound imaging transducer 270 includes one or more ultrasound elements. The module 200 can include one or more ultrasound imaging transducers 270 and/or one or more ultrasound therapy transducers 280. In some embodiments, an ultrasound therapy transducer 280 includes one or more ultrasound elements. The module 200 can include one or more ultrasound elements. In one embodiment, the module 200 comprises a bubble trap to reduce bubbles in an acoustic medium. The hand wand 100 can include or connect to imaging-only modules, treatment-only modules, imaging-and-treatment modules, and the like. In various embodiments, the ultrasound transducer 270 and/or 280 is movable in one or more directions 290 within the module 200. In some embodiments, the transducer 270 and/or 280 is connected to a motion mechanism 400. In some embodiments, the transducer 270 and/or 280 is not connected to a motion mechanism 400. In various embodiments, the motion mechanism can comprise one or more bearings, shafts 400 (e.g., rods, screws, lead screws), optional position sensing devices 402 (e.g., an encoder to measure position of the transducer), motors 403 (e.g., a step motor) to help ensure accurate and repeatable movement of the transducer within the module 200. In various embodiments, components in a motion mechanism can be in a module 200 and/or hand wand 100. In various embodiments, module 200 can include a transducer 270 and/or 280 which can emit energy through an acoustically transparent member 230. In one embodiment, the module 200 has an offset distance 210 between the transducer 270 and/or 280 and the acoustically transparent member 230. In one embodiment, the module 200 has an offset distance 211 between the imaging transducer 270 and bottom of an imaging region distance. In one embodiment, the console 300 comprises a control module that can be coupled to the hand wand 100 via the interface 130, and the graphic user interface 310 can be adapted to and/or configured for controlling the module 200. In one embodiment, the console 300 can provide power to the hand wand 100. In one embodiment, the hand wand 100 can include a power source. In one embodiment, the switch 150 can be adapted to and/or configured for controlling a tissue imaging function and the switch 160 can be adapted to and/or configured for controlling a tissue treatment function. In various embodiments, delivery of emitted energy 50 at a suitable focal depth, distribution, timing, and energy level is provided by the module 200 through controlled operation by the control system of the console 300 of the therapy transducer 280 to achieve the desired therapeutic effect with a thermal coagulation zone 550.

In one embodiment, the module 200 can be coupled to the hand wand 100. The module 200 can emit and receive energy, such as ultrasonic energy. The module 200 can be electronically coupled to the hand wand 100 and such coupling may include an interface which is in communication with the controller 305. In one embodiment, the interface guide 235 can be adapted to and/or configured to provide electronic communication between the module 200 and the hand wand 100. The module 200 can comprise various probe and/or transducer configurations. For example, the module 200 can be adapted to and/or configured for a combined dual-mode imaging/therapy transducer, coupled or co-housed imaging/therapy transducers, separate therapy and imaging probes, and the like. In one embodiment, when the module 200 is inserted into or connected to the hand wand 100, the controller 305 automatically detects it and updates the interactive graphical display 310.

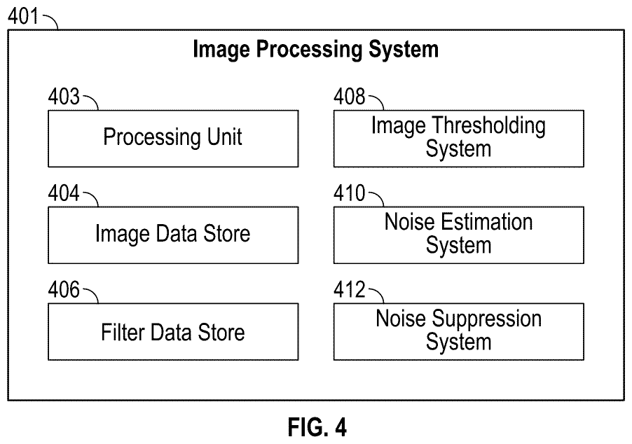

FIG. 4 illustrates an example block diagram of an image processing system 401, according to various embodiments of the present disclosure. In several embodiments, the image processing system 401 may be configured to process an ultrasound image captured by the ultrasound imaging system 20. Although not pictured in FIGS. 1-3, the image processing system 401 may be implemented in any of the components described above, such as the handpiece 100, transducer module 200, the console 300, or through any other related component or connected interface. In various embodiments, the image processing system 401 may be a single computing device, or it may include multiple distinct computing devices, such as computer servers, logically or physically grouped together to collectively operate as a system. In some embodiments, the features and services provided by the image processing system 401 may be implemented as web services consumable via a communication network. In further embodiments, the image processing system 401 is provided by one more virtual machines that are implemented in a hosted computing environment. The hosted computing environment may include one or more rapidly provisioned and released computing resources, which computing resources may include computing, networking and/or storage devices. A hosted computing environment may also be referred to as a cloud computing environment.

In one embodiment, the image processing system 401 may include a processing unit 403, an image data store 404, a filter data store 406, an image thresholding system 408, a noise estimation system 410, and a noise suppression system 412.

In one embodiment, processing unit 403 may include a processor (or multiple processors) that executes program instructions or modules stored in a memory or other non-transitory computer-readable storage medium or device (e.g., solid state storage devices, disk drives, etc.). For example, the processing unit 403 may be configured to execute any process, step, or task of the image processing system 401, such as to process an ultrasound image. For example, in some embodiments, the processing unit 403 may be configured to execute any process or task of the noise estimation system 410 and the noise suppression system 412. In some examples, the processing unit 403 may be communicatively coupled to other components of image processing system 401, such as the image data store 404, the filter data store 406, etc. The processing unit 403 may be configured to access image data stored in the image data store 404 for processing. In addition, the processing unit 403 may be configured to access filters, masks, or other algorithms stored in the filter data store 406 for processing of an image accessed from the image data store 404.

As shown in in FIG. 4, the image processing system 401 may include an image data store 404. In some embodiments, the image data store 404 may be configured to store ultrasonic images from the ultrasound imaging system 20. In some embodiments, the module 200 may be configured to visualize a tissue region and/or anatomy. In addition, the module 200 may capture an ultrasonic image of a visualized tissue region and/or anatomy. In some embodiments, the module 200 of the ultrasound imaging system 20 may capture a plurality of ultrasonic images, such as in the form of a 2D, 3D ultrasonic image, video, recording, etc. In one embodiment, each ultrasonic image of the recording or video may be a frame of the ultrasonic recording or video. In some examples, the image processing system 401 may be configured to access frames of an ultrasonic image from the image data store 404 for further processing. For example, the image processing system 401 may be configured to suppress debris noise that appears on the ultrasound image. Image data store 404 may include a random-access memory (RAM), cache and/or other dynamic storage devices, for storing information relating to the ultrasound imaging system 20.

The image processing system 401 may include filter data store 406. In some embodiments, the filter data store 406 may be configured to store algorithms, filters, functions, masks, etc. that may be accessed by processing unit 403 of the image processing system 401 to process ultrasonic frames. In some examples, the filter data store 406 may store noise suppression filters. In this example, the noise suppression filters may include a mask of any relevant pixel size. In some embodiments, the image processing system 401 may be configured to access a mask stored in the filter data store 406 to apply to a frame accessed from the image data store 404 to suppress noise (e.g., debris noise) from the ultrasonic image. In some embodiments, the mask may be used to convolve the ultrasonic image. In addition, the filter data store 406 may include any noise suppression filter, noise reduction filter, smoothing filter, low-pass filter, Sobel filter, etc. that may be used to suppress noise arising from debris in an ultrasonic image.

In some embodiments, the image thresholding system 408 may be configured to determine whether an ultrasonic image is suitable for further image processing. In the context of ultrasound imaging, there may be some images that are not suitable for processing due to inadequate contact with a patient's skin, hardware malfunctions, etc. The image thresholding system 408 may be configured to determine whether an image meets a threshold for further processing, such as noise suppression and/or noise estimation.

The noise estimation system 410 may be configured to estimate a level of noise present in an ultrasonic image of the ultrasound imaging system 20. The noise estimation system 410 may process an image after the image thresholding system 408 has determined that the image meets the threshold for further processing. In some embodiments, the noise estimation system 410 may access an ultrasonic image, such as an ultrasonic frame, stored in image data store 404. In addition, the noise estimation system 410 may estimate a level of noise present in the ultrasonic image accessed from the image data store 404. For example, the noise estimation system 412 may run one or more filters or functions on the ultrasonic image. In addition, the noise estimation system 410 may calculate a difference in contrast or other imaging value of an original ultrasonic image and a filtered ultrasonic image. The noise estimation system 410 may apply threshold processing and/or other gradient filters to determine an intensity of the ultrasonic image. It is noted that a variety of processes may be performed by the noise estimation system 410 to estimate a noise level of the ultrasonic image.

The noise suppression system 412 may be configured to suppress noise in an ultrasonic image of the ultrasound imaging system 20. The noise suppression system 412 may suppress debris noise of an ultrasonic image based on the estimated amount of noise, determined by the noise estimation system. In some embodiments, the noise suppression system 412 may be configured to suppress debris noise that appears on an ultrasound image. In some embodiments, the noise suppression system 412 may be configured to access an ultrasound image stored in the image data store 404. In addition, the noise suppression system 412 may be configured to access one or more masks, such as a noise suppression mask, stored in the filter data store 406. In some embodiments, the noise suppression system 412 may be configured to apply the mask to the ultrasound frame. In some embodiments, the image processing system 401 may apply the mask to the ultrasound frame or a number of iterations. For example, the mask may consist of a pixel mask (e.g., 3×3 pixel mask, 5×5 pixel mask) that may be passed over the ultrasonic image 1, 2, 3, 5, 7, 10, 15, 20 . . . N times.

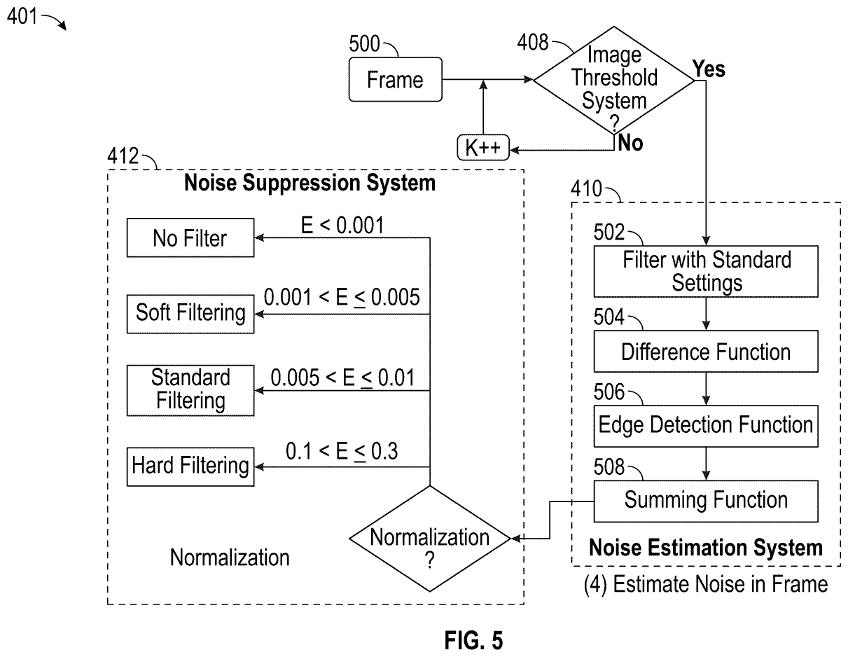

FIG. 5 illustrates an example block diagram of the components of image processing system 401 to process an ultrasonic image captured by the ultrasound imaging system 20. It is noted that the processes executed by the image processing system 401 may occur during an active ultrasound imaging session.

The image processing system 401 may receive and/or access a frame 500 corresponding to an ultrasonic image from the ultrasound imaging system 20. As noted herein, the ultrasound imaging system 20 may capture a plurality of ultrasonic images, such as in the form of a 2D, 3D ultrasonic image, video, recording, etc. In one embodiment, each ultrasonic image of the recording or video may be a frame of the ultrasonic recording or video. As such, the image processing system 401 may be configured to access frames of an ultrasonic image from the image data store 404 for further processing. For example, the image processing system 401 may receive or access a frame 500 of multiple frames of an ultrasonic image.

It is noted that the noise level assessments and filter parameter setting processes described herein may occur periodically during an active ultrasonic imaging session. For example, for every Nth frame received by the ultrasonic imaging system 20, the image processing system 401 may execute the various processes as described herein.

In some embodiments, the image thresholding system 408 may be configured to determine whether a frame 500, such as the Nth frame, of an ultrasonic image is suitable for further image processing. In the context of ultrasound imaging, there may be some images that are not suitable for processing due to inadequate contact with a patient's skin, hardware malfunctions, etc. For example, the image threshold system 408 may determine whether there is adequate contact between the module 200 and a patient's skin. The image thresholding system 408 may be configured to determine whether a frame of an ultrasonic image meets a threshold for further processing, such as noise estimation and/or noise suppression. It is noted that the processes and steps described may be performed by a processing unit 403 of the image processing system 401.

To evaluate whether or not a frame 500 is suitable for further noise estimation and suppression, the image thresholding system 408 may determine an intensity distribution over various areas of the frame. In some embodiments, the image thresholding system 408 may determine an absolute intensity of the frame 500. In some embodiments, if the absolute intensity is above a threshold value, the frame 500 is considered suitable for further processing by the noise estimation system 412 and/or the noise suppression system 410. In the case that the absolute intensity is below at or below a threshold value, the image thresholding system 408 may conduct additional processes on the ultrasonic image.

In some embodiments, the image thresholding system 408 may be configured to determine the intensity distribution over the area of the frame 500. For example, the image thresholding system 408 may divide, segment, or otherwise partition the frame 500 into separate areas. In addition, the image thresholding system 408 may compare an average intensity between each area in determining the intensity distribution over the area of the frame 500.

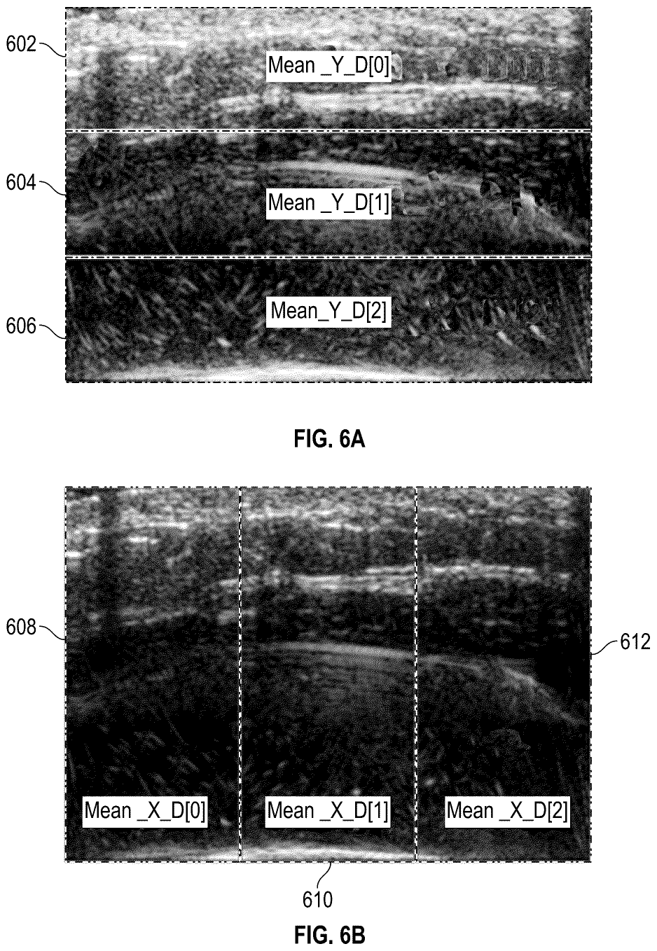

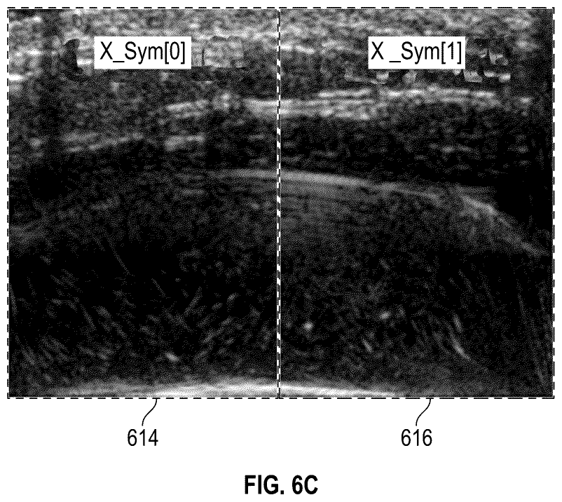

FIGS. 6A-6C illustrate portions of a sample frame 500 that may be processed by the image thresholding system 408, according to various embodiments of the present disclosure.

For example, as shown in FIG. 6A, the image thresholding system 408 may divide the frame 500 into a top row 602, middle row 604, and bottom row 606. In some embodiments, the frame 500 may be divided into more or fewer rows. In addition, the image thresholding system 408 may determine an intensity value, such as the average intensity, for the top row 602, middle row 604, and bottom row 606, respectively. As shown in FIG. 5A, the calculated intensity values for the top row 602, middle row 604, and bottom row 606 may be represented by arrays mean_Y_D[0], mean_Y_D[1], mean_Y_D[2], respectively. In some embodiments, the image thresholding system 408 may compare the intensity values of the rows (or other portions) to determine the intensity distribution of the frame 500. For example, the image thresholding system 408 may determine whether the intensity value of the top row 602 is greater than the middle row 604 and/or the bottom row 606. This may inform the image thresholding system 408 whether or not the frame 500 is a suitable image for further processing.

In an example, as shown in FIG. 6B, the image thresholding system 408 may divide the frame 500 into a left column 608, a middle column 610, and a right column 612. In addition, the image thresholding system 408 may determine an intensity value, such as the average intensity, for the left column 608, a middle column 610, and a right column 612, respectively. As shown in FIG. 6B, the calculated intensity values for the left column 608, a middle column 610, and a right column 612 may be represented by arrays mean_X_D[0], mean_X_D[1], mean_X_D[2], respectively. In some embodiments, the image thresholding system 412 may compare the intensity values of the rows (or other portions) to determine the intensity distribution of the frame 500. For example, the image thresholding system 408 may determine whether the intensity value of the left column 608 and the right column 612 is greater than the middle column 610. This may inform the image thresholding system 408 whether or not the frame 500 is a suitable image for further processing.

In an example, as shown in FIG. 6C, the image thresholding system 408 may divide the frame 500 into a left half 614 and a right half 616. In addition, the image thresholding system 408 may determine an intensity value, such as the average intensity, for the left half 614 and the right half 616, respectively. As shown in FIG. 5C, the calculated intensity values for the left half 514 and a right half 616 may be represented by arrays X_sym[0] and X_sym[1], respectively. In some embodiments, the image thresholding system 408 may compare the intensity values of the rows (or other portions) to determine the intensity distribution of the frame 500. For example, the image thresholding system 408 may determine whether the intensity value of the left half 614 and the right half 616 are approximately equal. This may inform the image thresholding system 408 whether or not the frame 500 is suitable image for further processing.

In the case when the image threshold system 408 determines that the frame 500 is not suitable for further processing, such as by the noise estimation system 410, the frame may be passed over. In addition, as shown in FIG. 5, a counter may be incremented in the case when the frame is determined to not be suitable.

In the case when the image threshold system 408 determines that the frame 500 is suitable for further processing, the frame may be transmitted to the noise estimation system 410.

The noise estimation system 410 may receive the frame 500 determined to be suitable (e.g., good contact between the module 200 and the patient's skin) from the image threshold system 408. The noise estimation system 410 may estimate a level of noise in the frame 500 before noise is suppressed by the noise suppression system 412. Alternatively, or in addition, the noise estimation system 410 may estimate a level of noise in the frame 500 after noise is suppressed by the noise estimation system 410. It is noted that the processes described with respect to the noise estimation system 410 and the noise estimation system 412 may be iterative. For example, the noise estimation system 410 may determine an initial level of noise in the frame 500 and the noise suppression system 412 may suppress the noise. The frame 500 may be inputting back into the noise estimation system 410 to estimate a remaining level of noise to determine whether additional iterations of the noise suppression mask(s) is needed.

As shown in FIG. 5, the noise estimation system 410 includes various filters or functions to process the frame 500 to estimate the level of noise. For example, the estimation process executed by the noise estimation system 410 may include a standard filter function 502, a difference function 504, an edge detection function 506, and a summing function 508. In some embodiments, more or less functions or filters may be applied to the frame 500 as a processing step within the noise estimation system 410.

The noise estimation system 410 may apply a standard filter function 502 to the frame 500. The standard filter function 502 may include any filter with parameters that are unadjusted or tuned to the frame 500 (e.g., initial settings). For example, the standard filter function 502 may include a 3×3 pixel mask or 5×5 pixel mask used to suppress low noise zones and/or small speckles in the ultrasonic image. The 3×3 pixel mask may be any noise suppression filter mask or standard mask, such as a smoothing or blurring mask, which may be used to reduce noise or artifacts in the frame 500. In some embodiments, the standard filter function 502 is a linear smoothing filter, in which each pixel of the frame 500 is replaced with an average of the neighboring pixels. In this case, the size of the neighboring pixels may be defined by the mask size. In some embodiments, the standard filter function 502 includes a nonlinear smoothing filter that replaces each pixel in the frame 500 with the median value of the neighboring pixels. For example, in some embodiments, the central value of the mask may be assigned an average value between the local maximum value and the local minimum value within the standard filter function 502. In addition, the noise estimation system 410 may apply the standard filter function 502 or multiple filters to the frame 500 multiple times. In response to applying the standard filter function 502 to the frame 500, the noise estimation system 410 may determine a filtered frame 500.

In some embodiments, the image processing system 104, such as via the noise estimation system 410, may divide, apportion, or otherwise determine zones of the frame 500 for application of various functions or filters. There may be varying levels of noise that may exist in the frame 500. To minimize loss of information, different filters or masks may be applied to the frame 500 in segmented areas based on the amount of detected noise. For example, an area of the frame 500 with smaller noise speckle size may require a smaller mask to smooth out or reduce the noise. In this case, a larger mask in this area may be unnecessary and over-process the ultrasound image. In some cases, an area with a larger noise speckle size may require a larger mask to smooth out or reduce the noise. For example, in this case, a small mask may not be able to adequately reduce the noise. As such, the noise estimation system 410 may apply different standard filter functions 502 to different zones of the frame 500. In addition, after the standard filter function 502 has been applied, the noise estimation system 410 may apply a difference function 504 to the frame 500. In some embodiments, the noise estimation system 412 may be configured to determine a difference between the frame 500 and the filtered frame 500. The noise estimation system 410 may determine a pixel-by-pixel difference between the frame 500 and the filtered frame 500. The noise estimation system 412 may be configured to determine the pixel-by-pixel difference by subtracting the filtered frame from the original frame 500. The pixel-by-pixel difference may represent the amount of noise filtered out by the application of the standard filter function 502 to the frame 500. In some examples, the noise estimation system 412 may determine a difference between the frame 500 and the filtered frame 500′ within a zone. The difference determined by the difference function 504 may be an absolute difference between the filtered frame and the frame 500. In response to the application of the difference function 504 to the filtered frame 500, the noise estimation system 410 may determine a difference frame 500.

After the noise estimation system 410 determines the difference frame 500, noise estimation system 410 may apply an edge detection function 506. Edge detection via the edge detection function 506 may highlight significant changes in image processing and may be used to detect any discontinuities in brightness or contrast. In this context, the edge detection function 506 may indicate whether additional processing and/or noise suppression is needed. For example, the noise estimation system 412 may apply any edge detection filter, such as a vertical filter (e.g., Sobel filter), a horizontal filter, and the like. In some examples, the filter may have an aperture size of 3×3 pixels. In some embodiments, the application of the edge detection filter to the pixel-by-pixel difference causes the noise estimation system 412 to output a gradient frame 500.

In response to the determination of a gradient frame 500 based on the application of the edge detection function 506, the noise estimation system 410 may apply a summing function 508. The summing function 508 may sum the results of the edge detection function 506, such as a vertical Sobel filter operation. In some embodiments, the noise estimation system 410 may be configured to determine an average intensity of the gradient frame 500. In some embodiments, the average intensity of the frame 500 may represent a level of noise estimation. In addition to summing the results of the edge detection function 506, the noise estimation system may normalize the normalize the average intensity (although in FIG. 5, the normalization decision block is shown to be within the noise suppression system 412). For example, the noise estimation system 410 may normalize the average intensity (“E”) of the gradient frame 500 to be a value within 0 to 1. In this example, an average normalized intensity closer to 0 indicates the presence of low noise, whereas an average normalized intensity closer to 1 indicates the presence of high noise.

In response to an estimated level of noise determined by the noise estimation system 410, the noise suppression system 412 may suppress debris noise from the frame 500 (and/or the gradient frame 500 as the output of the noise estimation system 410). In some embodiments, the amount of noise estimated by the noise estimation system 410 may be used to determine type of mask/filter to be applied to the frame 500.

As noted herein, the frame 500 may be partitioned into various zones. As such, the noise estimation system 410 and the noise suppression system 412 may determine a level of noise for each zone, and apply different filters to various zones according to the determination.

In one example, the noise estimation system 410 may determine that the normalized average intensity is below a value of 0.001. As such, the noise suppression system 412 may determine that little to no noise is present. In this case, the noise suppression system 412 may determine that no filter is needed for that frame 500 or for a zone of the frame 500.

In another example, the noise estimation system 410 may determine that the normalized average intensity is between 0.001 and 0.005 (e.g., 0.001<E≤0.005). Accordingly, the noise suppression system 412 may determine that a low level of noise is present, and a first filter should be applied to the mask. The first filter includes any noise suppression filter as described herein. In some cases, the noise suppression system 412 determines multiple iterations of the first filter to the frame 500 is needed. Based on the level of noise estimated per zone, the noise suppression system 412 may determine that additional iterations of the first filter are needed based on the zones.

In another example, the noise estimation system 410 may determine that the normalized average intensity is between 0.005 and 0.1 (e.g., 0.005<E≤0.005). Accordingly, the noise suppression system 412 may determine that a moderate amount of noise is present, and a second filter should be applied to the mask. The second filter includes any noise suppression filter as described herein, but configured to suppress a greater amount of noise than the first filter. In some cases, the noise suppression system 412 determines multiple iterations of the second filter to the frame 500 is needed. Based on the level of noise estimated per zone, the noise suppression system 412 may determine that additional iterations of the second filter are needed based on the zones.

In another example, the noise estimation system 410 may determine that the normalized average intensity is between 0.1 and 0.3 (e.g., 0.1<E≤0.3). Accordingly, the noise suppression system 412 may determine that a large amount of noise is present, and a third filter should be applied to the mask. The third filter includes any noise suppression filter as described herein, but configured to suppress a greater amount of noise than the first and second filter. In some cases, the noise suppression system 412 determines multiple iterations of the third filter to the frame 500 is needed. Based on the level of noise estimated per zone, the noise suppression system 412 may determine that additional iterations of the third filter are needed based on the zones.

In another example, the noise estimation system 410 may determine additional levels of noise present in the frame 500, and as such, additional filters may be applied to the frame 500 (not pictured in FIG. 5). For example, the noise estimation system 410 may determine that the normalized average intensity is greater than 0.3 (e.g., E>0.3). Accordingly, the noise suppression system 412 may determine that a very large amount of noise is present, and additional iterations of an additional filter to the frame 500 is needed.

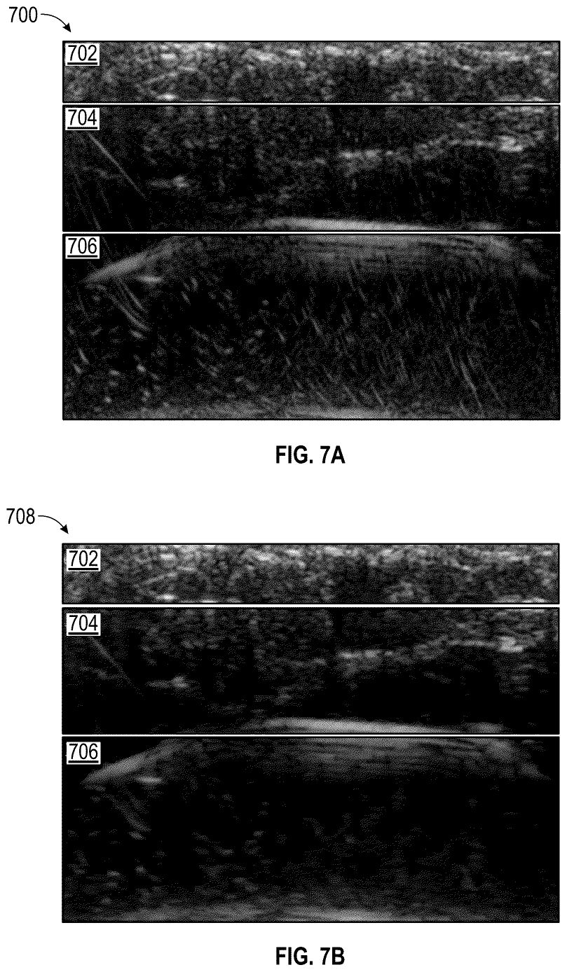

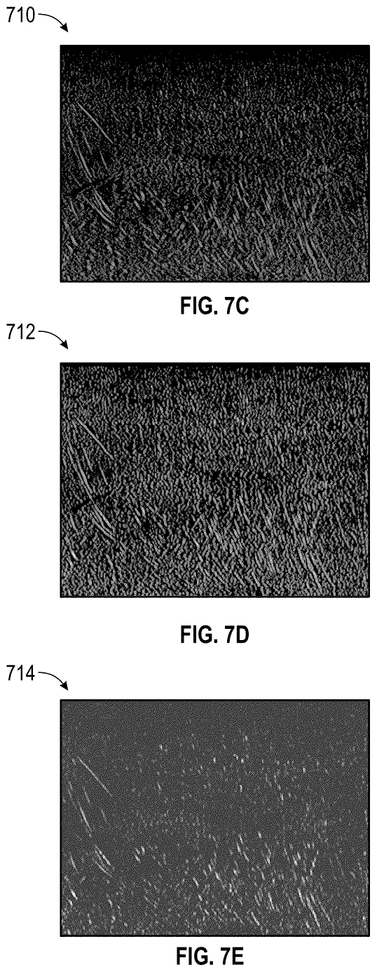

FIGS. 7A-7E illustrate a sample ultrasonic image 700 that may be processed by the noise estimation system 410 and the noise suppression system 412 as described in FIG. 5, according to one embodiment. As noted herein, debris, air bubbles, and/or other materials between the imaging element and the tissue region can interfere with the imaging and ultimately affect the image captured by the ultrasound imaging system. As shown in FIGS. 7A-7E, debris may appear on the ultrasonic image 700 as speckles or artifacts. In some embodiments, the noise estimation system 412 may estimate a level of noise in an original ultrasonic image 700 before noise is suppressed by the noise suppression system 410. In some embodiments, the noise estimation system 412 may estimate a level of noise in the ultrasonic image 700 after noise is suppressed by the noise suppression system 410. In one embodiment, the noise estimation system 412 may estimate a remaining level of noise in order to determine whether additional iterations of the noise suppression mask(s) is needed. In some embodiments, the image processing system 401 (e.g., noise suppression system 412 and/or noise estimation system 410) may divide, apportion, or otherwise determine zones of the ultrasonic image 700. As shown in FIGS. 7A and 7B, there are varying levels of noise that may exist in the ultrasonic image 700. To minimize loss of information, different filters or masks may be applied to the ultrasonic image 700 in segmented areas based on the amount of detected noise. For example, an area of the ultrasonic image 700 with smaller noise speckle size may require a smaller mask to smooth out or reduce the noise. In this case, a larger mask in this area may be unnecessary and over-process the ultrasound image. In some cases, an area with a larger noise speckle size may require a larger mask to smooth out or reduce the noise. For example, in this case, a small mask may not be able to adequately reduce the noise.

As shown in FIG. 7A, the ultrasonic image 700 may be divided or apportioned into three zones including a first zone 702, a second zone 704, and a third zone 706. In some examples, the ultrasonic image 600 may contain more or less than three zones. In some embodiments, a zone may include any portion of the area of ultrasonic image 700 and may be any shape or size. For example, a zone of the ultrasonic image 700 may include any shape, such as a rectangle, square, circle, oval, triangle, or another shape. In some embodiments, a zone may correspond to the entire area of the ultrasonic image 700. In some embodiments, the image processing system 401 may determine a zone on the ultrasonic image 700 based on the amount of noise.

As shown in FIG. 7A, the first zone 702 may be located at the top of the ultrasonic image 700. In some embodiments, the image processing system 401 may determine the first zone based on an amount of noise. For example, as shown in FIG. 7A, the top of the ultrasonic image 700 does not contain much noise, which may be due to the proximity to the module 200 and/or proximity to the surface of the tissue. As such, there may not be a need to filter the first zone 702. Accordingly, the noise estimation system 410 may omit filtering the ultrasonic image 700 with the standard filter function 502. In addition, the noise suppression system 412 may omit filtering in the first zone 702. The result of filtering by the standard filter function 502 is illustrated by FIG. 7B.

In some embodiments, the second zone 704 may correspond to an area of the ultrasonic image 700 that is non-overlapping with the first zone 702. In some embodiments, the second zone 704 may be located below the first zone 702 and may represent an area of the ultrasonic image 700 at a deeper depth than that of the first zone 702. As shown in FIG. 7A, the second zone 704 may have a low noise level and a small noise speckle size. In some embodiments, the second zone 704 may correspond to an area of the ultrasonic image 700 with a low noise level and/or a small speckle size. In some embodiments, the noise estimation system 410 and/or the noise suppression system 412 may apply a mask or filter, such as the standard filter function 502 etc., to the second zone 704 of the ultrasonic image 700. For example, the mask may include a noise suppression or smoothing function. In addition, the mask may be any relevant size, such as a 3×3 pixel or 5×5 pixel size. In some embodiments, the noise estimation system 410 and/or the noise suppression system 412 may apply the mask to the second zone 704 for a number of iterations. The number of iterations may be predetermined or preconfigured. For example, the number of iterations may be five times for a 3×3 pixel mask. In some embodiments, the number of iterations may correspond to a determined level of noise in the ultrasonic image 700 and/or the second zone 704.

In some embodiments, the third zone 706 may correspond to an area of the ultrasonic image 600 that is non-overlapping with the first zone 702 or the second zone 704. In some examples, the third zone 706 may be located below the second zone 704 and may represent an area of the ultrasonic image 700 at a deeper depth than that of the first zone 702 or the second zone 704. As shown in FIG. 7A, the third zone 706 may have a high noise level and a large noise speckle size. In some embodiments, the third zone 706 may correspond to an area of the ultrasonic image 700 with a high noise level and/or a large noise speckle size. In some embodiments, the noise estimation system 410 and/or the noise suppression system 412 may apply a mask or filter, such as the standard filter function 502 etc., to the third zone 706 of the ultrasonic image 700. For example, the mask may include a noise suppression or smoothing function. In addition, the mask may be any relevant size, such as a 3×3 or 5×5 pixel size. It is noted that the mask size of the third zone may be larger or smaller than the mask used in a different zone of the ultrasonic image 700 to accommodate the relative speckle size in the zone. In some embodiments, the noise suppression system 410 may apply the mask to the third zone 706 for a number of iterations. The number of iterations may be predetermined or preconfigured. For example, the number of iterations may be seven times for a 5×5 pixel mask. In some embodiments, the number of iterations may correspond to a determined level of noise in the ultrasonic image 700 and/or the third zone 706.

In some embodiments, the image processing system 401 may determine additional zones on the ultrasonic image 700. For example, the image processing system 401 may determine sub-zones within the zones 702, 704, 706, or determine additional zones at greater depth, etc.

FIG. 7A illustrates an example source ultrasonic image 700 that may be received by the image threshold system 408. Image threshold system 408 may process the received ultrasonic image 700 according to the processes described with respect to FIG. 5. The image threshold system 408 may determine that the ultrasonic image 700 meets a threshold for further processing. In response to applying the standard filter function 502 to the ultrasonic image 700, the noise estimation system 410 may determine a filtered ultrasonic image 708. FIG. 7B illustrates the filtered ultrasonic image 708 processed by the noise estimation system 410 with standard filter function 502 as described with respect to FIG. 5. In this example as shown, the ultrasonic image 700 may result after five interactions with a standard filter function consisting of a 3×3 pixel mask in the second zone 704 and seven iterations with a 5×5 pixel mask in the third zone 706. It is noted that the speckles, such as in the third zone 706, may be reduced due to the iterations of the 5×5 pixel mask in the third zone 706.

FIG. 7C illustrates the filtered ultrasonic image 708 processed by the noise estimation system 410 with an applied difference function 504 as described with respect to FIG. 5. As described herein, the noise estimation system 410 may be configured to determine a difference between the original ultrasonic image 700 and the filtered ultrasonic image 708. FIG. 7C illustrates a pixel-by-pixel difference between the original ultrasonic image 700 and the filtered ultrasonic image 708. In some embodiments, the noise estimation system 410 may be configured to determine the pixel-by-pixel difference by subtracting the filtered ultrasonic image 708 from the original ultrasonic image 700. In some embodiments, the pixel-by-pixel difference may represent the amount of noise filtered out by the application of the mask(s) to the original ultrasonic image 700. In some examples, the noise estimation system 410 may determine a difference between the original ultrasonic image 700 and the filtered ultrasonic image 708 within a zone. For example, the noise estimation system 412 may determine a pixel-by-pixel difference by subtracting the filtered ultrasonic image 708 in the third zone 706 from the original ultrasonic image 700 in the third zone 706. FIG. 7D illustrates a gradient ultrasonic image 712 resulting from the application of the edge detection function to the difference ultrasonic image 710. In some embodiments, the noise estimation system 410 may be configured to apply an edge detection function 506 to the pixel-by-pixel difference. Edge detection may highlight significant changes in image processing and may be used to detect any discontinuities in brightness or contrast. In this context, the edge detection may indicate whether additional processing and/or noise suppression is needed. For example, the noise estimation system 410 may apply any edge detection filter, such as a vertical filter (e.g., Sobel filter), a horizontal filter, and the like. In some examples, the filter may have an aperture size of 3×3 pixels. In some embodiments, the application of the edge detection filter to the pixel-by-pixel difference causes the noise estimation system 410 to output a gradient ultrasonic image 710. FIG. 7D illustrates the output gradient ultrasonic image 712 resulting from the application of the edge detection function 506 to the

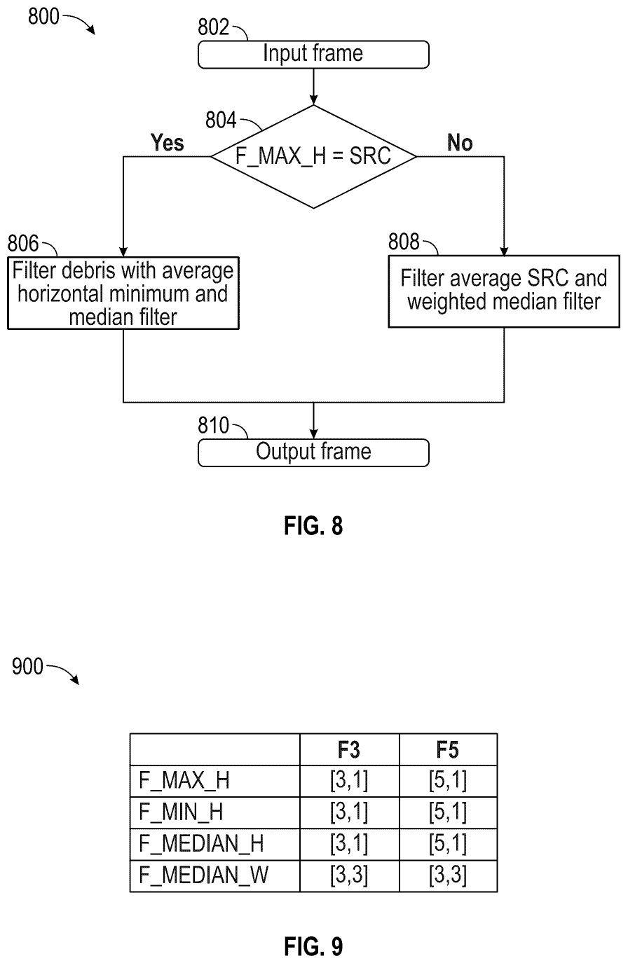

FIG. 7E illustrates a summed ultrasound image 714 resulting from the application of the summing function 508 to the gradient ultrasonic image 712. In some embodiments, the noise estimation system 412 may be configured to determine an average intensity of the gradient ultrasonic image 712. As shown in FIG. 7E, the average intensity of the summed ultrasound image 714 may be illustrated. In some embodiments, the average intensity of the summed ultrasound image 714 may represent a level of noise estimation. In some embodiments, the noise estimation system 412 may perform threshold processing on the summed ultrasound image 714. For example, the noise estimation system 412 may not take into account small or miniscule changes in the intensity of the summed ultrasound image 714, such as changes that may not be related to the specific noise. In addition, the noise estimation system 412 may normalize the average intensity. For example, the noise estimation system 412 may normalize the average intensity (“E”) of the summed ultrasound image 714 to be a value within 0 to 1. In this example, an average intensity closer to 0 indicates the presence of low noise, whereas an average intensity closer to 1 indicates the presence of high noise. FIG. 8 illustrates an example flow diagram of debris detection and filtering logic utilized by the image processing system 401. The processes described in FIG. 8 may be applied by the image processing system 401 (e.g., noise estimation system 410, noise suppression system 412) with respect to the application of a filter or mask.

As described herein, the image processing system 401 may apply filter(s) or mask(s) to an input ultrasonic image or frame of an ultrasonic image, such as a noise suppression mask. In some examples, the standard filter function 502 may include a noise suppression mask as described herein. In addition, masks accessed by the noise suppression system 412 may include a noise suppression mask as described herein. The noise suppression mask may be a smoothing or blurring mask, which may be used to reduce noise or artifacts in the ultrasonic image. Alternatively, or in addition, the noise suppression mask may be a linear smoothing filter, in which each pixel is replaced with an average of the neighboring pixels. In this case, the size of the neighboring pixels may be defined by the mask size. In some embodiments, the noise suppression filter includes a nonlinear smoothing filter that replaces each pixel in the image with the median value of the neighboring pixels, depending on the filter size. For example, in some embodiments, the central value of the mask may be assigned an average value between the local maximum value and the local minimum value within the mask. In some embodiments, the ultrasonic image may be convolved with the noise suppression filter. The size of the noise suppression mask may be used to suppress or filter noise or speckles of different sizes. For example, a 3×3 pixel mask may be used to suppress low noise zones and/or small speckles in the ultrasonic image. In some embodiments, a 5×5 pixel mask may be used to suppress high noise zones and/or large speckles in the ultrasonic image.

As described below, the flow diagram 800 may depict filtration utilizing a 3×3 or 5×5 filter to an ultrasound image to suppress debris noise. To do so, a portion of a filter to suppress bright outliers may be adapted to the size of the bright outlier's cross section along the x-axis. Specifically, the flowchart may represent an algorithm executed by the image processing system 401 based on a comparison of a source pixel value (“SRC”) and the result of a horizontal maximum filter (“F_MAX_H”).

At step 802, an input frame of an ultrasonic image is received by the image processing system 401. Each pixel of the input frame may be processed by image processing system 401 according to the flow diagram 800. At step 804, the image processing system 401 may determine, for a first pixel of the input frame, whether the SRC is equal to the result of an applied F_MAX_H. As noted herein, debris noise may be characterized by vertically and obliquely directed curves and lines of high intensity. These lines usually have a projection on the X-axis that is significantly smaller than the projection on the Y-axis. As such, the horizontal cross-section of these high-intensity lines typically consists of no more than a few pixels. Accordingly, at 804, the image processing system 401 may determine suspected debris presence when the output value of the horizontal max filter is equal to the SRC.

If the F_MAX_H is equal to the SRC, the image processing system 401 may proceed in the flow diagram 800 to step 806. At step 806, the image processing system 401 may determine a destination pixel value (“DST”) based on an averaging of a horizontal minimum filter (F_MIN_H) and the median filter (“F_Median_H”).

If the F_MAX_H is not equal to the SRC, the image processing system 401 may proceed in the flow diagram 800 to step 808. At step 808, the image processing system 401 may determine a DST based on an averaging of the SRC and the median filter (“F_Median_H”). AT block 808, this process deals with small filtering of a part of the image in which there is no noise debris.

Steps 804-808 may be performed iteratively for each pixel of the input frame. In some cases, when each pixel of the input frame has been processed by the image processing system 104 according to the flow diagram 800, the image processing system 104 may output the output frame 810.

FIG. 9 is a table 900 illustrating example filter aperture sizes. Table 900 includes the various types of filters/masks used by the image processing system 104, including a horizontal maximum filter, a horizontal minimum filter, a horizonal median filter, and a vertical median filter. As shown by the columns, each filter may be various sizes. The F3 filter designation may correspond to a 3×1 of 3×3 pixel size. The F5 filter designation may correspond to a 5×1 of 5×5 pixel size.

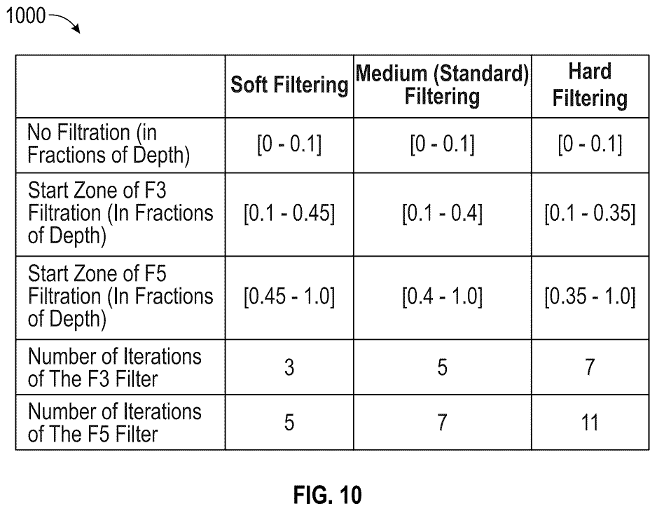

FIG. 10 is a table illustrating example filters and corresponding depth ranges and iterations. Depending on the type of filtering (e.g., soft, medium, hard), the image processing system 401 may apply either F3 or F5 filter at various depths and iterations as shown.

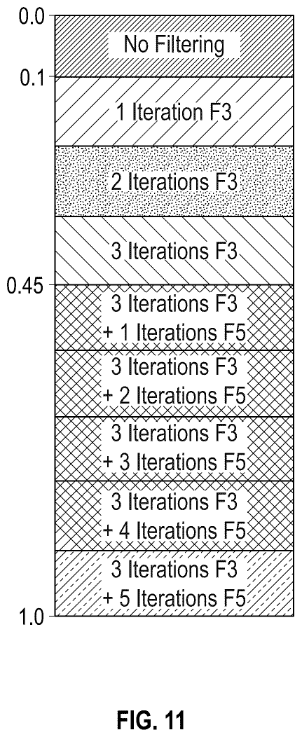

FIG. 11 is an example scheme of filter sizes and iterations used by the image processing system at corresponding zones of an ultrasonic image.

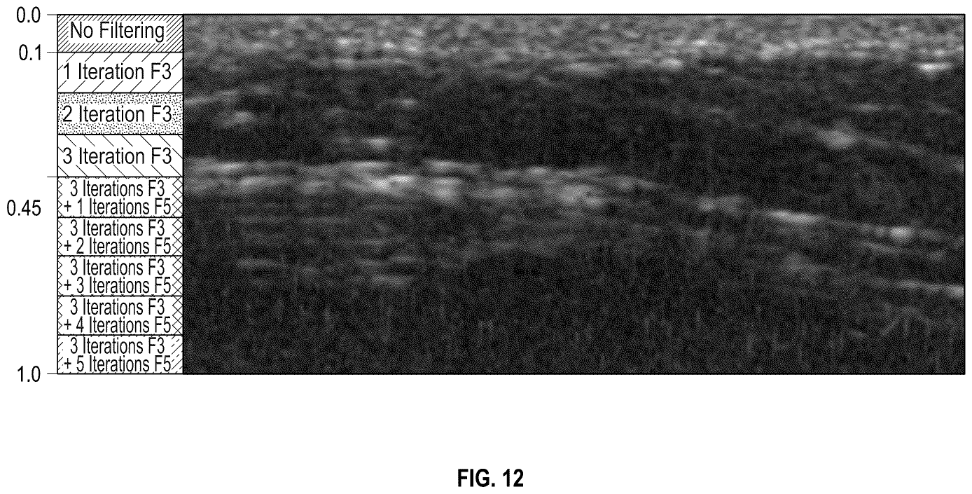

FIG. 12 is an example scheme of filter sizes and iterations used by the image processing system at corresponding zones of an ultrasonic image containing weak debris noise.

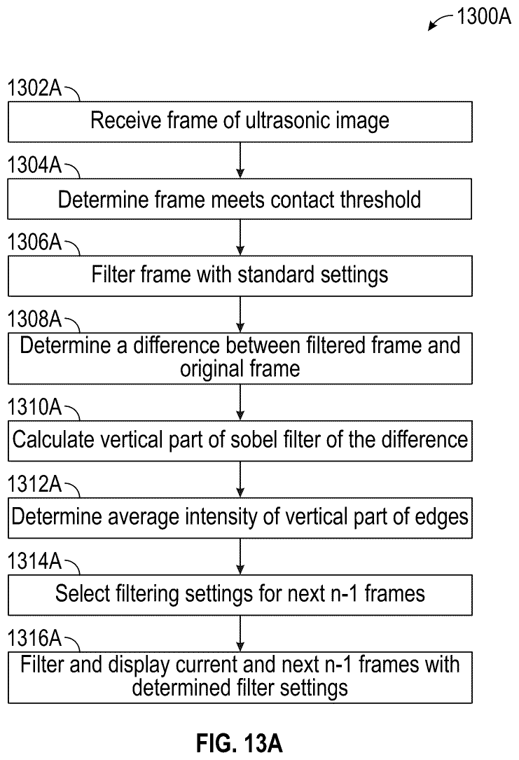

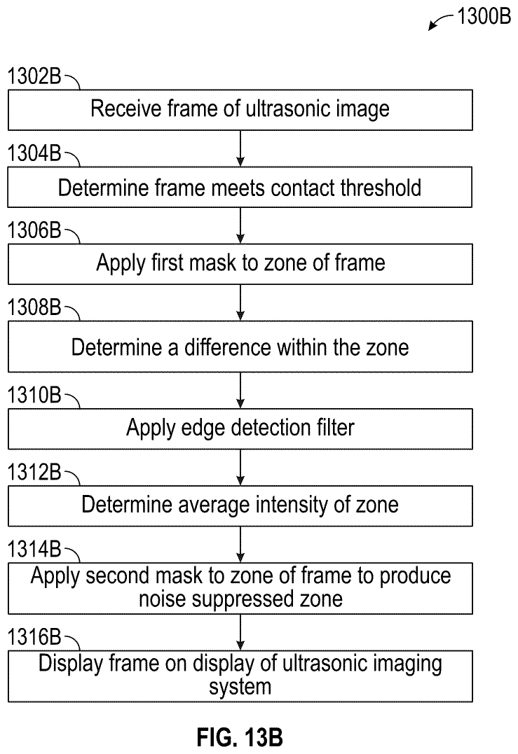

FIG. 13A is an example flow diagram of a routine 1300A to digitally filter noise debris from an ultrasonic image of an ultrasound imaging system 20. The routine 1300A may be executed by the processing until 403 of the image processing system 401 and the various components of the image processing system 401.

At block 1302, a frame 500 of an ultrasonic image is received by the image processing system 401. The ultrasonic image may be from the ultrasound imaging system 20. As noted herein, the ultrasound imaging system 20 may capture a plurality of ultrasonic images, such as in the form of a 2D, 3D ultrasonic image, video, recording, etc. In one embodiment, each ultrasonic image of the recording or video may be a frame of the ultrasonic recording or video. As such, the image processing system 401 may be configured to access frames of an ultrasonic image from the image data store 404 for further processing. For example, the image processing system 401 may receive or access a frame 500 of multiple frames of an ultrasonic image.

At block 1304, the image thresholding system 408 may determine that the frame meets a contact threshold. To determine whether the frame meetings the contact threshold, the image thresholding system 408 may determine whether the frame 500, such as the Nth frame, of an ultrasonic image is suitable for further image processing. In the context of ultrasound imaging, there may be some images that are not suitable for processing due to inadequate contact with a patient's skin, hardware malfunctions, etc. For example, the image threshold system 408 may determine whether there is adequate contact between the module 200 and a patient's skin. The image thresholding system 408 may be configured to determine whether a frame of an ultrasonic image meets a contact threshold for further processing, such as noise estimation and/or noise suppression.

The contact threshold may correspond to an intensity distribution of the frame. To evaluate whether or not a frame 500 is suitable for further noise estimation and suppression, the image thresholding system 408 may determine an intensity distribution over various areas of the frame. In some embodiments, the image thresholding system 408 may determine an absolute intensity of the frame 500. In some embodiments, if the absolute intensity is above a threshold value, the frame 500 is considered suitable for further processing by the noise estimation system 412 and/or the noise suppression system 410. In the case that the absolute intensity is below at or below a threshold value, the image thresholding system 408 may conduct additional processes on the ultrasonic image. In some embodiments, the image thresholding system 408 may be configured to determine the intensity distribution over the area of the frame 500. For example, the image thresholding system 408 may divide, segment, or otherwise partition the frame 500 into separate areas. In addition, the image thresholding system 408 may compare an average intensity between each area in determining the intensity distribution over the area of the frame 500.

At block 1306A, the image processing system 104 filters the frame 500 with standard settings. As used herein, standard settings may include the medium (standard) filtering values as shown in the corresponding column in FIG. 10.

At block 1308A, the image processing system 104 determines or calculates a difference between the frame 500 as entirely filtered and the original frame 500.

At block 1310A, the image processing system 104 may calculate a vertical part of the Sobel filter from the difference obtained in block 1308 of the frame 500.

At block 1312A, the image processing system 104 determines an average intensity of the vertical part of the Sobel filter (e.g., edges) which was obtained in block 1310.

At block 1314A, filtering settings are selected for the current frame 500 and the next N−1 frames. The threshold values for selecting filtering settings are shown in FIG. 5. Filtering zones (e.g., in fractions of image depth) and the number of iterations for each type of filtering are shown in FIG. 10.

At block 1316A, the image processing system 104 filters and displays the frame and next N−1 frames with determined filter settings on a display of the ultrasound imaging system.

FIG. 13B is an example flow diagram of a routine 1300B to digitally filter noise debris from an ultrasonic image of an ultrasound imaging system 20. The routine 1300 may be executed by the processing unit 403 of the image processing system 401 and the various components of the image processing system 401.

At block 1302B, a frame 500 of an ultrasonic image is received by the image processing system 401. The ultrasonic image may be from the ultrasound imaging system 20. As noted herein, the ultrasound imaging system 20 may capture a plurality of ultrasonic images, such as in the form of a 2D, 3D ultrasonic image, video, recording, etc. In one embodiment, each ultrasonic image of the recording or video may be a frame of the ultrasonic recording or video. As such, the image processing system 401 may be configured to access frames of an ultrasonic image from the image data store 404 for further processing. For example, the image processing system 401 may receive or access a frame 500 of multiple frames of an ultrasonic image.

At block 1304B, the image thresholding system 408 may determine that the frame meets a contact threshold. To determine whether the frame meetings the contact threshold, the image thresholding system 408 may determine whether the frame 500, such as the Nth frame, of an ultrasonic image is suitable for further image processing. In the context of ultrasound imaging, there may be some images that are not suitable for processing due to inadequate contact with a patient's skin, hardware malfunctions, etc. For example, the image threshold system 408 may determine whether there is adequate contact between the module 200 and a patient's skin. The image thresholding system 408 may be configured to determine whether a frame of an ultrasonic image meets a contact threshold for further processing, such as noise estimation and/or noise suppression.

The contact threshold may correspond to an intensity distribution of the frame. To evaluate whether or not a frame 500 is suitable for further noise estimation and suppression, the image thresholding system 408 may determine an intensity distribution over various areas of the frame. In some embodiments, the image thresholding system 408 may determine an absolute intensity of the frame 500. In some embodiments, if the absolute intensity is above a threshold value, the frame 500 is considered suitable for further processing by the noise estimation system 412 and/or the noise suppression system 410. In the case that the absolute intensity is below at or below a threshold value, the image thresholding system 408 may conduct additional processes on the ultrasonic image. In some embodiments, the image thresholding system 408 may be configured to determine the intensity distribution over the area of the frame 500. For example, the image thresholding system 408 may divide, segment, or otherwise partition the frame 500 into separate areas. In addition, the image thresholding system 408 may compare an average intensity between each area in determining the intensity distribution over the area of the frame 500.

At block 1306B, the image processing system 104 applies a first mask to a zone of the frame. In some embodiments, the image processing system 104, such as via the noise estimation system 410, may divide, apportion, or otherwise determine zones of the frame 500 for application of various functions or filters. In addition, at block 1306, the image processing system 104, such as via the noise estimation system 410, may apply a first mask to the zone. The first mask includes the standard filter function 502, may include any filter with parameters that are unadjusted or tuned to the frame 500 (e.g., initial settings). For example, the first mask may include a 3×3 pixel mask or 5×5 pixel mask used to suppress low noise zones and/or small speckles in the ultrasonic image. The 3×3 pixel mask may be any noise suppression filter mask or standard mask, such as a smoothing or blurring mask, which may be used to reduce noise or artifacts in the frame 500. In some embodiments, the first mask is a linear smoothing filter, in which each pixel of the frame 500 is replaced with an average of the neighboring pixels. In this case, the size of the neighboring pixels may be defined by the mask size. In some embodiments, the first mask includes a nonlinear smoothing filter that replaces each pixel in the frame 500 with the median value of the neighboring pixels. For example, in some embodiments, the central value of the mask may be assigned an average value between the local maximum value and the local minimum value within the standard filter function 502. In addition, the noise estimation system 410 may apply the first mask or multiple masks to the frame 500 multiple times.

At block 1308B, the image processing system 104 determines a difference within the zone. In some embodiments, the image processing system 104 determines a difference between the frame and the frame with the first mask applied. To determine a difference, the noise estimation system 410 may apply a difference function 504 to the frame 500. In some embodiments, the noise estimation system 412 may be configured to determine a difference between the frame 500 and the filtered frame 500. The noise estimation system 410 may determine a pixel-by-pixel difference between the frame 500 and the filtered frame 500. The noise estimation system 412 may be configured to determine the pixel-by-pixel difference by subtracting the filtered frame from the original frame 500. The pixel-by-pixel difference may represent the amount of noise filtered out by the application of the standard filter function 502 to the frame 500. In some examples, the noise estimation system 412 may determine a difference between the frame 500 and the filtered frame within a zone. The difference determined by the difference function 504 may be an absolute difference between the filtered frame and the frame 500. In response to the application of the difference function 504 to the filtered frame 500, the noise estimation system 410 may determine a difference frame.

At block 1310B, the image processing system 104 applies an edge detection filter to the zone of the frame. For example, the noise estimation system 410 may apply an edge detection function 506. Edge detection via the edge detection function 506 may highlight significant changes in image processing and may be used to detect any discontinuities in brightness or contrast. In this context, the edge detection function 506 may indicate whether additional processing and/or noise suppression is needed. For example, the noise estimation system 412 may apply any edge detection filter, such as a vertical filter (e.g., Sobel filter), a horizontal filter, and the like. In some examples, the filter may have an aperture size of 3×3 pixels. In some embodiments, the application of the edge detection filter to the pixel-by-pixel difference causes the noise estimation system 412 to output a gradient frame 500.

At block 1312B, the image processing system 104 determines an average intensity of the zone. The average intensity may correspond to an estimated level of noise within the zone. the noise estimation system 410 may apply a summing function 508. The summing function 508 may sum the results of the edge detection function 506, such as a vertical Sobel filter operation. In some embodiments, the noise estimation system 410 may be configured to determine an average intensity of the gradient frame 500. In some embodiments, the average intensity of the frame 500 may represent a level of noise estimation. In addition to summing the results of the edge detection function 506, the noise estimation system may normalize the normalize the average intensity (although in FIG. 5, the normalization decision block is shown to be within the noise suppression system 412). For example, the noise estimation system 410 may normalize the average intensity of the gradient frame 500 to be a value within 0 to 1. In this example, an average normalized intensity closer to 0 indicates the presence of low noise, whereas an average normalized intensity closer to 1 indicates the presence of high noise.

At block 1314B, the image processing system 104 applies, based on the estimated level of noise in the zone, a second mask to the zone to produce a noise suppressed zone of the frame. The second mask includes the standard filter function 502, may include any filter with parameters that are unadjusted or tuned to the frame 500 (e.g., initial settings). For example, the second mask may include a 3×3 pixel mask or 5×5 pixel mask used to suppress low noise zones and/or small speckles in the ultrasonic image. The 3×3 pixel mask may be any noise suppression filter mask or standard mask, such as a smoothing or blurring mask, which may be used to reduce noise or artifacts in the frame 500. In some embodiments, the second mask is a linear smoothing filter, in which each pixel of the frame 500 is replaced with an average of the neighboring pixels. In this case, the size of the neighboring pixels may be defined by the mask size. In some embodiments, the second mask includes a nonlinear smoothing filter that replaces each pixel in the frame 500 with the median value of the neighboring pixels. For example, in some embodiments, the central value of the mask may be assigned an average value between the local maximum value and the local minimum value within the standard filter function 502. In addition, the noise estimation system 410 may apply the second mask or multiple masks to the frame 500 multiple times.

In some embodiments, application of the first or second mask comprises determining, for each pixel of the zone of the frame, whether an input pixel value is equal to a result of a horizontal maximum filter, determining an output pixel value based on a weighted median filter to the pixel in response to the determination that the input pixel value is equal to the result of the horizonal maximum filter, and determining an output pixel value based on an average of the horizontal maximum filter and a horizontal minimum filter in response to the determination that the input pixel value is not equal to the result of the horizonal maximum filter.

At block 1316B, the image processing system 104 displays the frame on a display of the ultrasound imaging system.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules, including one or more specific computer-executable instructions, that are executed by a computing system. The computing system may include one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of electronic devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. A computing environment can include any type of computer system, including, but not limited to, a computer system based on a microprocessor, a mainframe computer, a digital signal processor, a portable electronic device, a device controller, or a computational engine within an appliance, to name a few.

Conditional language such as, among others, “can,” “could,” “might,” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures. Should be understood as potentially representing modules, segments, or portions of code which include one or more executable instructions for implementing specific logical functions or elements in the process. Alternate embodiments are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

1. An ultrasound imaging system configured for digitally filtering debris from an ultrasonic image, comprising:
an ultrasonic probe comprising an ultrasound imaging transducer configured for imaging a tissue region;
a display coupled to the ultrasonic probe; and
a processor coupled to the ultrasonic probe and configured to:
receive a frame of an ultrasonic image from the ultrasound imaging transducer;
apply a first mask to the frame to produce a difference frame, determine an absolute difference between the frame and the frame with the first mask applied;
apply an edge detection filter to the difference frame;
determine an average intensity of the difference frame, wherein the average intensity corresponds to an estimated level of remaining noise within the frame;
apply, based on the estimated level of remaining noise, a second mask to the frame to produce a noise suppressed frame; and
send a signal to display the noise suppressed frame on the display.

2. The ultrasound imaging system of claim 1, wherein the first mask and the second mask are noise suppression masks.

3. The ultrasound imaging system of claim 1, wherein the processor is configured to normalize the estimated level of remaining noise in the frame.

4. The ultrasound imaging system of claim 1, wherein the processor is further configured to apply additional iterations of the second mask to the noise suppressed frame.

5. The ultrasound imaging system of claim 1, wherein the processor is configured to:
determine that additional noise suppression is needed based on the estimated level of noise; and
apply additional iterations of the second mask to the frame.

6. The ultrasound imaging system of claim 1, wherein application of the first mask or the second mask further causes the processor to:
determine, for a pixel of the frame, whether an input pixel value is equal to a result of a horizontal maximum filter;
determine a first output pixel value based on a weighted median filter to the pixel in response to a determination that the input pixel value is equal to the result of the horizontal maximum filter; and
determine a second output pixel value based on an average of the horizontal maximum filter and a horizontal minimum filter in response to a determination that the input pixel value is not equal to the result of the horizontal maximum filter.

7. The ultrasound imaging system of claim 1, further comprising a coupling medium configured to acoustically couple the ultrasound imaging transducer to a surface of the ultrasonic probe.

8. The ultrasound imaging system of claim 1, wherein the ultrasound imaging transducer is configured to image at least one of an epidermal tissue, a dermal tissue, and a subdermal tissue.

9. The ultrasound imaging system of claim 1, further comprising one or more ultrasound therapy transducers configured for a treatment in the tissue region.

10. The ultrasound imaging system of claim 9, wherein the treatment is a face lift, a wrinkle reduction, a décolletage improvement, a buttock lift, or a skin tightening.

11. An ultrasound imaging system configured for digitally filtering debris from an ultrasonic image, comprising:
an ultrasonic probe comprising an ultrasound imaging transducer configured for imaging a tissue region;
a display coupled to the ultrasonic probe; and
a processor coupled to the ultrasonic probe and configured to:
apply a first filter to an ultrasonic image,
determine an absolute difference between the ultrasonic image and the ultrasonic image with the first filter applied to produce a difference image;
apply an edge detection filter to the difference image;
determine an intensity of the difference image, wherein the intensity corresponds to an estimated level of remaining noise of the ultrasonic image;
apply, based on the estimated level of remaining noise, a second filter to the ultrasonic image to produce a noise suppressed ultrasonic image; and
send a signal to display the noise suppressed ultrasonic image on the display.

12. The ultrasound imaging system of claim 11, wherein the first filter and the second filter are noise suppression filters.

13. The ultrasound imaging system of claim 11, wherein the processor is further to normalize the estimated level of remaining noise.

14. The ultrasound imaging system of claim 11, wherein the processor is further configured to apply additional iterations of the second filter to the ultrasonic image.

15. The ultrasound imaging system of claim 11, wherein the processor is further configured to:
determine that additional noise suppression is needed based on the estimated level of noise; and
apply additional iterations of the second filter to the ultrasonic image.

16. The ultrasound imaging system of claim 11, wherein application of the first filter or the second filter further causes the processor to:
determine, for a pixel of the ultrasonic image, whether an input pixel value is equal to a result of a horizontal maximum filter;
determine a first output pixel value based on a weighted median filter to the pixel in response to a determination that the input pixel value is equal to the result of the horizontal maximum filter; and
determine a second output pixel value based on an average of the horizontal maximum filter and a horizontal minimum filter in response to a determination that a source pixel value is not equal to the result of the horizontal maximum filter.

17. The ultrasound imaging system of claim 11, further comprising a coupling medium configured to acoustically couple the ultrasound imaging transducer to a surface of the ultrasonic probe.

18. The ultrasound imaging system of claim 11, wherein the ultrasound imaging transducer is configured to image at least one of an epidermal tissue, a dermal tissue, and a subdermal tissue.

19. The ultrasound imaging system of claim 11, further comprising one or more ultrasound therapy transducers configured for treating a tissue in the tissue region.

20. The ultrasound imaging system of claim 11, further comprising one or more ultrasound therapy transducers configured for a face lift, a wrinkle reduction, a décolletage improvement, a buttock lift, or a skin tightening.
